(19) United States
(12) Patent Application Publication
(10) Pub. No.: US 2008/0182853 A1
(43) Pub. Date: Jul. 31, 2008

(54) Methods of neuroprotection by cyclin-dependent kinase inhibition

(76) Inventors: Inna Kruman, Lubbock, TX (US); Elena Schwartz, Derwood, MD (US)

Correspondence Address: PANACEA PHARMACEUTICALS, INC., 207 PERRY PARKWAY, SUITE 2, GAITHERSBURG, MD, 20877, US

(21) Appl. No.: 11/957,392

(22) Filed: Dec. 14, 2007

Related U.S. Application Data
(60) Provisional application No. 60/874,844, filed on Dec. 14, 2006.

(51) Int. Cl. A61K 31/496; A61P 25/00; A61P 9/00; A61P 25/28

(52) U.S. Cl. 514/252.16

(57) ABSTRACT

The present invention relates to methods of suppressing neuronal death, such as is observed with ischemia-related diseases and disorders, including neuronal and cardiac conditions arizing from a sudden loss of oxygen and/or energy loss, and degenerative diseases, such as Alzheimer's disease to name just one. The methods involve the use of inhibitors that act primarily in a simultaneous manner on the cyclin-dependent kinases, CDK4 and CDK6.

This application claims priority to U.S. provisional application Ser. No. 60/874,844, filed Dec. 14, 2006, the entire contents of which are hereby incorporated by reference.

### FIELD OF THE INVENTION

The present invention relates to methods of suppressing neuronal death, such as seen with so-called ischemia-related diseases and disorders, including for example neuronal and cardiac diseases due to sudden loss of oxygen, as well as longer-term degenerative diseases, such as Alzheimer's disease among others. The methods involve the use of inhibitors that act primarily in a simultaneous manner on the cyclin-dependent kinases, CDK4 and CDK6, an example of which is the compound, PD0332991 (Pfizer).

### BACKGROUND OF THE INVENTION

The present invention is broadly directed to a new use of certain cyclin-dependent kinase (CDK) inhibitors, more particularly inhibitors of CDK4 and CDK6 together (CDK4/6), which have until now been shown useful only as antineoplastic agents. Such CDK4/6 inhibitors and their syntheses have been disclosed inter alia in U.S. Pat. No. 6,396,612. The present inventors have found that such compounds also have the characteristic of acting as neuroprotectants, and as such are useful in acute and chronic nervous system disorders and conditions and other diseases and disorders in which ischemia plays an essential role in the pathology.

Increasing evidence indicates that maintenance of neuronal homeostasis involves the activation of the cell cycle machinery in postmitotic neurons. Recently, the present inventors' studies have suggested that cell cycle activation is essential for DNA damage-induced neuronal apoptosis Accumulating evidence suggests that activation of the cell cycle machinery contributes to the demise of terminally differentiated neurons exposed to damaging stimuli (Kiruman, I. I., et al. 2004. Neuron. 41:549-561; Liu, D. X., and L. A. Greene. 2001. Cell Tissue Res. 305:217-228). In mitotic cells, the cell cycle machinery is a major contributor to the DNA damage response, acting through a complex set of mechanisms that repair the damage and coordinate cell division and apoptosis in a collective effort to preserve genomic integrity (Abraham, R. T. 2003. Bioessays. 25:627-630; Bernstein. C., et al. 2002. Mutat Res. 511:145-178; Rhind, N. and P. Russell. 2000. Curr Biol. 10:R908-R911). Accordingly, the processes of cell cycle regulation and DNA repair are functionally integrated, as evidenced by the fact that they use several common proteins (Slupphaug, G., et al. 2003. Mutat Res. 531:231-251). The protection of genomic integrity is a major challenge for cells, which are continuously exposed to genotoxic stress resulting from exogenous sources and from endogenous free radicals that arise from oxygen metabolism. Neurons are highly susceptible to oxidative stress due to their high rate of oxidative metabolism and low level of antioxidant enzymes (Brooks, P. J. 2000. Neurochem Int. 37:403-412). Consequently, oxidative stress represents a major cause of the neuropathology underlying a variety of neurodegenerative diseases (Sayre, L. M., et al. 2001. Curr Med Chem. 8:721-738). DNA damage is an important initiator of neuronal death in a wide variety of neuropathological conditions (Bogdanov, M., et al. 2000. Free Radic Biol Med. 29:652-658; Jenner, P. and C. W. Olanow. 1998. Ann Neurol. 44(3 Suppl 1):S72-84; Lovell, M., et al., 1999. J. Neurochem. 72:771-776). A connection between DNA damage and neurodegeneration is also illustrated by the neurological abnormalities that accompany defective DNA repair in various human syndromes such as ataxia telangiectasia and Cockayne syndrome (Rolig, R. L., and P. J. McKinnon. 2000. Trends Neurosci. 23:417-424).

Terminally differentiated neurons are transcriptionally active and retain the need to preserve the integrity of the transcribed genome throughout the life span, underscoring the importance of an adequate DNA damage response in these cells. Thus, the high metabolic rate and continuous exposure to oxidative stress make the control of genomic integrity a challenging but essential task for postmitotic neurons, as evidenced by the fact that defects in the DNA damage response lead to severe neurodegeneration.

While a number of studies have investigated the responses of proliferating cells to genotoxic agents, the DNA damage response in terminally differentiated neurons is poorly understood.

Research has shown that cell cycle activation plays an essential role in neuronal death. The suppression of cyclin-dependent kinases (CDKs), critical for cell cycle progression, is known to be neuroprotective in experimental models of stroke (Johnson K, et al. (2005). J. Neurochem. 93:538-548; Katchanov, J., et al. 2001. J. Neurosci. 21:5045-5053; Kruman, I. I., et al. 2004. Neuron. 41:549-561). For example, flavopiridol, a non-specific CDK inhibitor that inhibits all the CDKs, has been shown to be very potent in preventing neuronal apoptosis in vitro, and was protective in in vivo ischemia models (Ginsberg D. (2002). FEBS Lett. 529:122-125; Knockaert M, et al. 2002. Trends Pharmacol. Sci. 23:417-425).

However, the mechanism of the neuroprotection has not been well understood. The present inventors attribute this to that fact that prior work with CDK inhibitors has been done with agents that are not very specific to certain CDK complexes. For instance, flavopiridol has multiple cellular targets, including non-CDK-related kinases (Fry, D. W., et al. 2004. Mol Cancer Ther. 3:1427-1438).

The present invention elucudates for the first time that the specific inhibition of the CDK4/6 kinases is sufficient for preventing neuronal apoptosis. It has been hypothesized that the DNA damage response and associated apoptotic signaling in neurons are linked to cell cycle activation. While not being bound by a particular theory, with the present invention it is thought that neuroprotection occurs in neurons due to the action of a CDK4/6 inhibiting agent targeting and inhibiting cell cycle activation and, consequently, apoptosis.

The present invention thus provides a method for protecting neurons under exogenenous or physiological stress, and accordingly provides methods for treating acute and chronic neurological disease states, by the inhibition of CDK4/6.

While the present invention is primarily directed to agents that act on both CDK4 and CDK6 together, an agent that acts to inhibit one of these is alone is contemplated to be included herein.

Recently, we and others have shown that the DNA damage response in postmitotic neurons committed to apoptosis involves cell cycle-associated events (Klein, J. A., et al. 2002. Nature. 419:367-374; Kruman, I. I., et al. 2004. Neuron. 41:549-561). While these observations are in keeping with the notion that resting cells must activate cell cycle machinery in response to DNA damage to eliminate cells with non-repairable damage, the present invention, i.a., provides further evidence that the cell cycle machinery is involved in apoptotic signaling in postmitotic neurons.

### SUMMARY OF THE INVENTION

Cyclin-dependent kinases (CDKs) are a family of serine/threonine protein kinases that regulate cell cycle progression upon complexing with their corresponding cyclin partner (Vermeulen, K., et al. 2003. Cell Prolif. 36:165-175). In general, pharmacological inhibition of CDK activity results in selective anti-proliferative effects on cycling cells (Gray, N., et al. 1999. Curr Med Chem. 6:859-875).

The neuronal effects of CDK4/6 inhibitors was discovered in the course of studying the DNA damage response of neurons under stress conditions. In neurons, mounting data suggest that the CDK/pRb/E2F pathway plays a prominent role in promoting neuronal cell death, and that CDK inhibitors have a neuroprotective effect (Katchanov, J., et al. 2001. J. Neurosci. 21:5045-5053; Meijer, L. and E. Raymond. 2003. Acc Chem Res. 36:417-425; Park, D. S., et al. 2000. Neurobiol Aging. 21:771-781). However, if the cell cycle machinery is involved in DNA repair, CDK suppression should block it.

This hypothesis was tested by employing RNAi directed against CDK4 and CDK6, two CDKs that are essential for cell cycle activation. By these studies, the present inventors demonstrated that the simultaneous inhibition of CDK4 and CDK6 activity actually blocked apoptosis, suggesting an important role for CDK4 and CDK6 in apoptotic signaling in postmitotic neurons. Thus, the data confirm the involvement of the cell cycle machinery in the neuronal apoptosis initiated by DNA damage.

Cell division cycle machinery is involved in the activation of the apoptotic cascade to eliminate cells that have incurred DNA damage (Bernstein. C., et al. 2002. Mutat Res. 511:145-178; Rhind, N. and P. Russell. 2000. Curr Biol. 10:R908-R911). The data presented here suggest that in postmitotic, terminally differentiated neurons, signaling through cell cycle components is also essential for the response to DNA damage; however, in contrast to cycling cells, which undergo growth arrest at specific checkpoints, DNA damage signaling in neurons is associated with activation of the cell cycle machinery.

This distinct response of neurons is thought to reflect the unique involvement of G1 cell cycle components in the activation of the neuronal DNA repair machinery.

While no pharmacological agents for neuroprotection are currently marketed, there are drugs approved for use in the therapy of chronic neurological conditions, which are glutamate receptor (NMDA) antagonists. Although there is evidence of ameliorating affects of such drugs in chronic CNS degenerative states, it does not appear that NMDA antagonists, alone, can provide substantial protection against ischemia, generally, especially in an acute situation.

A significant limitation of glutamate receptor antagonists as neuroprotectants against ischemic neurodegeneration is that they appear to insulate the neuron against degeneration only temporarily; they do not do anything to correct the energy deficit, or to correct other derangements that occur secondary to the energy deficit. Therefore, although these agents do provide some level of protection against ischemic neurodegeneration, the protection is only partial and in some cases may only be a delay in the time of onset of degeneration.

Since neurons begin to degenerate very rapidly after the onset of acute conditions such as ischemic injury, there is clearly a need for therapeutic agents that will actively protect neurons from further degeneration and death by, for example, suppressing apoptotic signaling. Such therapeutic agents could not only be used for acute instances of ischemia, but also preventing neuronal degeneration in chronic degenerative disorders, such as Alzheimer's and Parkinson's diseases on the basis of slowing down neuronal loss and neuronal degeneration.

Further, the compounds of the present invention can also be used to treat neurological disorders of the ear and eye that result from ischemic-like etiology, as well as diabetic neuropathies.

The development of therapeutic agents capable of preventing or treating the consequences of neuronal stress, whether acute or chronic, is highly desirable.

The present invention relates to methods of preventing and/or treating disorders resulting from neuronal stress conditions by administering to a patient in need of such treatment certain CDK4/6 inhibitors, such as PD 0332991, and pharmaceutically acceptable salts or prodrugs thereof:

The present invention is also directed to methods of treating, ameliorating, and/or preventing specific neuronal stress or ischemia-related conditions, including but not limited to treatment of neuronal damage following global and focal ischemia from any cause (and prevention of further ischemic damage), treatment or prevention of otoneurotoxicity and of eye diseases involving ischemic conditions (such as macular degeneration), prevention of ischemia due to trauma or coronary bypass surgery, treatment or prevention of neurodegenerative conditions such as amyotrophic lateral sclerosis (ALS), Alzheimer's disease, Parkinson's disease, and Huntington's chorea, and treatment or prevention of diabetic neuropathies.

### BRIEF DESCRIPTION OF THE DRAWINGS

FIG. 1: Apoptotic death of cultured cortical neurons is induced by 100 μM but not 5 μM H₂O₂.

(A) Cultures were exposed for 24 h to either saline (Con), 5 μM H₂O₂ or 100 μM H₂O₂ and then stained with DNA-binding dye propidium iodide (PI). Note the nuclear DNA condensation and fragmentation in cultures exposed to 100 μM H₂O₂.

(B) Cultures were exposed to either saline (Con), 5 μM H₂O₂, or 100 μM H₂O₂ during the indicated time periods and the dynamics of apoptosis in the cell populations were determined. The values are the means and SD (n=6); *p<0.01; **p<0.001.

(C) Immunoblot showing cleaved caspase-3 (19- and 17-kDa intermediates), fractin (cleaved β-actin; 32-kDa intermediate), cleaved Mcm3 (98-kDa intermediate), and non-cleaved Mcm2 in primary cortical neurons after the indicated time periods following exposure to either 5 or 100 μM H₂02. Control (C) corresponds to untreated cultures. An extract from methyl methanesulfonate (MMS)-treated NIH/3T3 cells was included as positive control. Note the appearance of cleaved caspase-3, Mcm3 and 13-actin (fractin) in samples exposed to 100 μM H₂O₂.

FIG. 2: DNA damage induced by H₂02.

(A) Cultures were exposed to either 5 μM or 100 μM H₂O₂ for the indicated periods of time. DNA damage was quantified by alkaline or neutral comet analysis in cortical neurons after the indicated time periods of incubation. Control (Con) represents untreated cells. As a positive control we used neurons exposed to 1 Gy of γ-irradiation. Note the higher levels of DNA damage in alkaline compared with neutral comet assay (different scales). Values are the means and SEM of determinations made in 3 cultures; *p<0.005; **p<0.001; #p<0.01; ##p<0.002.

(B) Micrographs showing immunoreactivity for γ-H2AX in cortical neurons treated for 6 h with vehicle (Con), 5 μM and 100 μM H₂O₂ and visualized with FITC (488, green). Cells were co-stained with PI. Note the induction of γ-H2AX foci in cultures exposed to 5 μM and 100 μM before significant apoptotic death (6 h) in contrast to control culture.

FIG. 3: A significant reduction in the extent of apoptosis in cells with silenced CDK4 and CDK6 expression.

(A) Successful knockdown of CDK4 and CDK6 expression by co-transfecting CDK4 and CDK6-specific siRNAs is shown by Western blot analysis.

(B) CDK4 and CDK6 expression in cortical neurons was knocked down, and susceptibility to 100 μM H₂O₂ induced cell death (18 h of exposure) was studied. Neurons transfected with non-specific control RNAi and those treated with 100 μM H₂O₂ were used as controls. Apoptosis was quantified in cortical cultures stained with Hoechst 33342 by calculating apoptotic nuclei. The values are the mean and SD (n=6); **p<0.001.

FIG. 4: Pharmacological suppression of CDK4 and CDK6 by PD 0332991 significantly reduces the extent of apoptosis in cortical neurons treated with H₂O₂

(A) PD 0332991 down-regulates phosphorylation of pRb in postmitotic neurons. Cultured cortical neurons were exposed either to saline, or 100 μM H₂O₂ alone or after 12 h pretreatment with 1 μM PD 0332991 (PD) for 6 h and the pRB phosphorylation was determined by Western blot analysis. Control (Con)—untreated culture; G1—HeLa cells synchronized in G1 phase of the cell cycle.

(B) Apoptosis was quantified in cortical cultures exposed for 18 h to 100 μM H₂O₂ after staining with Hoechst 33342 by calculating apoptotic nuclei. The values are the mean and SD (n=6); *p<0.002.

### DETAILED DESCRIPTION OF THE INVENTION

The present invention provides for therapeutic treatment, amelioration or prevention of neuronal degeneration and/or neuronal death in acute or chronic conditions, whereby a subject in need thereof is administered an therapeutically effective amount of an agent that acts as an inhibitor of one or both of CDK4 and CDK6. Such an agent may act by interference of RNA or as a small molecule pharmacological drug. In a preferred embodiment, the agent is an inhibitor of both CDK4 and CDK6.

It is demonstrated herein that the specific suppression of CDK4 and CDK6 by RNA interference, or pharmacologically by PD 0332991(Pfizer), significantly reduces the extent of apoptosis in primary cortical neurons exposed to hydrogen peroxide. The results show that the suppression of CDK4 and CDK6 is sufficient for neuroprotection in vitro. The molecule, PD 0332991, has been shown to be effective in causing tumor regression in mice, and is currently being used in human clinical trials for cancer. The structure of PD 0332991 is:

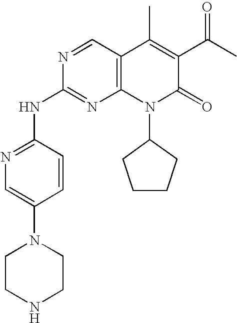

The present inventors have determined that, as a highly specific pharmacological inhibitor of CDK4 and CDK6 (aka “CDK 4/6”), the compound PD 0332991 exerts a neuroprotective effect in an oxidative DNA damage model of apoptosis due to the suppression of cell cycle reentry of neurons, essential for activating the apoptotic signaling. In this regard, PD 0332991 (a most preferred embodiment), as well as other agents that exhibit similar specificity in the inhibition of CDK4/6, have usefulness as therapeutic agents in such acute conditions as stroke, as preventatives in such instances as cardiac by-pass surgery, and as ameliorators or inhibitors of the progression of chronic neurological conditions, such as Alzheimer's, Parkinson's and ALS.

More particularly, the present invention contemplates that agents such as the exemplified PD 0332991 are useful in the treatment of the underlying ischemic causes of such diseases and conditions as: Alzheimer's disease; Parkinson's disease; ischemic states that are due to or result from such conditions as coronary artery bypass graft surgery; global cerebral ischemia due to cardiac arrest; focal cerebral infarction; cerebral hemorrhage; hemorrhage infarction; hypertensive hemorrhage; hemorrhage due to rupture of intracranial vascular abnormalities; subarachnoid hemorrhage due to rupture of intracranial arterial aneurysms; hypertensive encephalopathy; carotid stenosis or occlusion leading to cerebral ischemia; cardiogenic thromboembolism; spinal stroke and spinal cord injury; diseases of cerebral blood vessels (such as atherosclerosis and vasculitis); macular degeneration and other eye diseases such as retinopathies and glaucoma; myocardial infarction; cardiac ischemia; or superaventicular tachyarrhythmia. This list is not exhaustive, and one skilled in the art would understand that this invention is applicable to many physical ailments in which a physiologically ischemic condition prevails in the etiology—i.e., that a neuronal (esp.) or other cellular degeneration/cell death is at the root of the disease process, whether acute or chronic.

As described below, the present inventors investigated whether G1 (cell cycle) phase components contribute to the repair of DNA and are involved in the DNA damage response of postmitotic neurons. In terminally differentiated cortical neurons, treatment with toxic concentrations of hydrogen peroxide (H₂O₂) caused non-repairable DNA double-strand breaks (DSBs) and the activation of G1 components of the cell cycle machinery. Importantly, neuronal apoptosis was attenuated if cyclin-dependent kinases CDK4 and CDK6, essential elements of G0→G1 transition, were suppressed. Our data suggest that G1 cell cycle components are involved in the DNA response and DNA damage-initiated apoptisis of postmitotic neurons.

With the present invention, it was shown that the cell cycle machinery is a key component of the DNA damage response and apoptotic signaling of postmitotic neurons. To show this, the present inventors investigated the effects of toxic concentrations of H₂O₂ on postmitotic cortical neurons. The data indicate that oxidative stress elicited by exposure to toxic concentrations of H₂O₂ induced the formation of non-repairable DSBs associated with activation of cell cycle machinery and neuronal apoptosis. Apoptosis was attenuated if the essential G1 cell components CDK4 and CDK6 were suppressed.

Such results are indicative of a way to therapeutically treat subjects (humans or other animals) to inhibit, ameliorate or prevent damage to cells, a particularly significant subset of which are neurons.

Thus, from the disclosure of the present invention, it will be apparent to the skilled artisan that agents such as those disclosed herein can be administered in a pharmaceutically and therapeutically appropriate manner to a patient in need of such intervention, whereby the patient is physically and clinically assisted in overcoming the effects of cell degeneration and cell death (esp. neuronal), and the patient's condition is ameliorated and (further) damage prevented.

It is surprising and unexpected that a compound such as PD 0332991 (and similarly acting compounds) is effective as a neuroprotectant against ischemic cellular insult, given that its only known use thus far has been proposed as an antineoplastic agent.

Thus, one of the embodiments of the present invention is directed to the amelioration of the effects of ischemic cellular insult, particular on nerve cells/tissue. The present invention also contemplates the prophylactic administration of compounds such as PD 0332991 in subjects suspected of a familial or genetic risk for developing a chronic neurodegenerative condition, such as Alzheimer's or Parkinson's disease.

Compounds useful in the present invention, CDK4/6 inhibitors (such as PD 0332991), and their syntheses have been disclosed inter alia in U.S. Pat. No. 6,396,612, and well known in the art.

In a further aspect, the invention is directed to pharmaceutical compositions of the CDK4/6 inhibitors (such as PD 0332991) useful in the methods of the invention. The pharmaceutical compositions of the invention comprise one or more of the compounds (or one of the compounds together with one or more different active ingredients) and a pharmaceutically acceptable carrier or diluent. As used herein “pharmaceutically acceptable carrier or diluent” includes any and all solvents, dispersion media, solid excipients (e.g., binders, lubricants, etc. typically used in solid oral dosage forms) coatings, antibacterial and antifungal agents, isotonic and absorption delaying agents, and the like that are physiologically compatible. The type of carrier can be selected based upon the intended route of administration.

In various embodiments, the carrier is suitable for intravenous, intraperitoneal, subcutaneous, intramuscular, topical, transdermal or oral administration. For example, pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. In all dosage forms, supplementary active compounds may be incorporated into the compositions as well.

Preferably, administration is oral, and may be of an immediate or delayed release. Such oral pharmaceutical compositions of the present invention are manufactured by techniques typically used in the pharmaceutical industry. Generally, the active agent(s) is/are preferably formulated into a tablet or capsule for oral administration, prepared using methods known in the art, for instance wet granulation and direct compression methods. The oral tablets are prepared using any suitable process known to the art. See, for example, Remington's Pharmaceutical Sciences, 18th Edition, A. Gennaro, Ed., Mack Pub. Co. (Easton, Pa. 1990), Chapters 88-91, the entirety of which is hereby incorporated by reference. Typically, the active ingredient, i.e., one or more of the CDK4/6 inhibitors, is mixed with pharmaceutically acceptable excipients (e.g., the binders, lubricants, etc.) and compressed into tablets. Preferably, such a dosage form is prepared by a wet granulation technique or a direct compression method to form uniform granulates. Alternatively, the active ingredient(s) can be mixed with a previously prepared non-active granulate. The moist granulated mass is then dried and sized using a suitable screening device to provide a powder, which can then be filled into capsules or compressed into matrix tablets or caplets, as desired.

In one such aspect, the tablets are prepared using a direct compression method. The direct compression method offers a number of potential advantages over a wet granulation method, particularly with respect to the relative ease of manufacture. In the direct compression method, at least one pharmaceutically active agent and the excipients or other ingredients are sieved through a stainless steel screen, such as a 40 mesh steel screen. The sieved materials are then charged to a suitable blender and blended for an appropriate time. The blend is then compressed into tablets on a rotary press using appropriate tooling.

Alternatively, the pharmaceutical composition is contained in a capsule containing beadlets or pellets. Methods for making such pellets are known in the art (see, Remington's, supra). The pellets are filled into capsules, for instance gelatin capsules, by conventional techniques.

Sterile injectable solutions can be prepared by incorporating a desired amount of the active compound in a pharmaceutically acceptable liquid vehicle and filter sterilized. Generally, dispersions may be prepared by incorporating the active compound into a sterile vehicle containing a basic dispersion medium. In the case of sterile powders for the preparation of sterile injectable solutions, the preferred methods of preparation are vacuum drying and freeze-drying, which will yield a powder of the active ingredient plus any additional desired ingredient from a previously sterile-filtered solution thereof.

The pharmaceutical compositions of the present invention may be administered by any means to achieve their intended purpose, for example, by oral, parenteral, subcutaneous, intravenous, intramuscular, intraperitoneal, transdermal, or buccal routes.

The active agent(s) in the pharmaceutical composition (i.e., one or more of the CDK-4/6 inhibitors) is present in a therapeutically effective amount. By a “therapeutically effective amount” is meant an amount effective, at dosages and for periods of time necessary, to achieve the desired therapeutic result of positively influencing the course of a particular disease state or acute condition. This terminology also contemplates and encompasses the therapeutic use of the compounds in a prophylactic manner, which may be of a lower dosage, and such an embodiment is included in the present invention. Of course, therapeutically effective amounts of the active agent(s) may vary according to factors such as the disease state, age, sex, and weight of the individual, and the ability of the agent to elicit a desired response in the individual. Dosage regimens may be adjusted to provide the optimum therapeutic response. A therapeutically effective amount is also one in which any toxic or detrimental effects of the agent are outweighed by the therapeutically beneficial effects.

The amount of active compound in the composition may vary according to factors such as the disease state, age, sex, and weight of the individual. Dosage regimens may be adjusted to provide the optimum therapeutic response. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals. It is contemplated that the dosage units of the present invention will contain the active agent(s) in amounts suitable for a dosage regimen of about the same as or, more preferably less than, those derivable from the studies in the Example, which are thought to be effective below its maximal tolerated dose.

The pharmaceutical compositions of the invention may be administered to any animal in need of the beneficial effects of the compounds of the invention. While the invention is primarily directed to human use, other mammals in which an ischemic disease or condition is suspected may be treated accordingly if so desired.

This invention is further illustrated by the following example, which is not intended to limit the present invention. The contents of all references, patents, and published patent applications cited throughout this application are specifically and entirely incorporated herein by reference.

### EXAMPLE

### Cortical Cell Cultures and Experimental Treatments

All experiments involving the use of animals were approved by the IACUC at the Georgetown University Medical Center, Washington, D.C. Primary cortical cell cultures were established from E18 Sprague-Dawley rats obtained from Jackson Laboratories.

The cells were plated according to procedures described earlier (Kruman, I. I., et al. 2004. Neuron. 41:549-561). Following dissociation by mild trypsinization and trituration, cells were seeded onto plastic dishes or chamber slides precoated with 0.025 μg/ml poly-L-lysine, at a density of 1.3×10³ neurons/mm² in Neurobasal medium containing B-27 supplement, 1 mM HEPES, 2 mM glutamate and 0.001% gentamycin sulfate; fresh medium was replaced after 30 minutes.

All of the experiments were performed with 4-day-old cultures, a time during which ˜3% of the MAP-2-positive cells were in S phase (Kiruman, I. I., et al. 2004. Neuron. 41:549-561). A fresh stock of 1 mM hydrogen peroxide (H₂O₂; Sigma) was prepared in Neurobasal medium for each experiment and added at the indicated concentrations (5 μM and 100 μM). Treatment with 1 μM PD 0332991 (obtained from Pfizer) was carried out for 12 h in complete medium; H₂O₂ was added at the indicated times and doses.

Analysis of neuronal survival and apoptosis. Neuronal viability was assessed by quantifying apoptotic nuclei following the treatments. Cells were fixed and stained with DNA-binding dye propidium iodide (PI) (10 μg/ml; Sigma), and the percentage of cells with apoptotic nuclei was calculated as described previously (Kruman, I. I., et al. 2002. J. Neurosci. 22:1752-1762; Tenneti, L. and S. A. Lipton. 2000. J Neurochem. 74:134-142). Nuclear staining was viewed and photographed using a Zeiss fluorescence microscope. Apoptosis was also determined by immunoblot analysis for activated (cleaved) caspase-3 (polyclonal; 1 μg/ml; Upstate Cell Signaling Solutions), cleaved Mcm3 (polyclonal, 1:200; Santa-Cruz), Mcm2 (BM28; monoclonal; 1:200; BD Biosciences), and fractin (cleaved β-actin; 1:3000; Chemicon) in cellular extracts from corresponding neuronal 5 cultures. Extracts from methyl methanesulfonate (MMS)-treated NIH/3T3 cells were used as a positive control (Lakin, N. D. and S. P. Jackson. 1999. Oncogene. 18:7644-7655).

Immunoblot analyses. For total cell lysates, cortical neurons were lysed in ice-cold buffer consisting of 63 mM Tris, 2 mM EDTA, 2 mM EGTA, 2% sodium dodecyl sulfate, 10% glycerol, and a protease inhibitor cocktail (Sigma), pH 6.0. For the preparation of nuclear lysates (for analysis of cyclin D1 and γ-H2AX), cortical neurons were lysed in ice-cold buffer containing protease inhibitor cocktail (Sigma) and incubated with hydrochloric acid (0.2 M) on ice for 30 min.

After centrifugation, the acid-insoluble pellet was discarded and the supernatant was dialyzed against 200 ml 0.1 M acetic acid twice (1-2 h each time) and then dialyzed against water. As a positive control, we used extracts from HeLa cells synchronized in the G1 phase of the cell cycle (G1). HeLa cells were synchronized in mitosis by adding 0.1 μg/ml nocodazole. After 12 h, the mitotic cells were replated in nocodazole-free medium, and G1 cells were collected 3-5 hours later. Synchronization was monitored by flow cytometry.

Proteins (50 μg/lane) were size-separated by SDS-PAGE (10-15%), transferred to nitrocellulose membranes, and incubated for 30 min in the presence of 5% nonfat milk and incubated overnight at 4° C. with primary antibodies recognizing either γ-H2AX (monoclonal; 1 μg/ml; Upstate Cell Signaling Solutions), phospho RB at Ser 795 (polyclonal; 1:1000; Cell Signaling Technoogy), Rb (monoclonal, 1:2000; Cell Signaling Technology), Mcm3 (polyclonal; 1:200; Santa Cruz), Mcm2 (BM28 monoclonal; 1:500; BD Biosciences), cleaved caspase-3 (polyclonal; 1:1000; Cell Signaling Technology), and-fractin (cleaved β-actin; C-terminus polyclonal antibody; 1:3000; Chemicon). As a loading control we used anti-β-actin or anti-glyceraldehyde-3-phosphate dehydrogenase (GAPDH) antibodies.

Protein bands were visualized with horseradish peroxidase-conjugated secondary antibodies (1:3000; Jackson Immunological Research Laboratories Inc.) and enhanced using chemiluminescence (ECL kit, Amersham Corp.). Densitometric analysis of the blots were performed using the Kodak software ID 3.5.3 USB, and the intensity of the signal (normalized to the β-actin signals) was expressed as a ratio of the control signals.

siRNA preparation and transfection. siRNA oligonucleotides targeting CDK4 and CDK6 (SMARTpool reagents, Dharmacon), each representing a cocktail of four siRNAs directed against different regions of corresponding genes, were designed and used according to the manufacturer's guidelines. siRNA oligonucleotides targeting MAPK1 (Qiagen) was used as a control siRNA. Double-stranded siRNAs were generated by mixing the corresponding mixture of siRNA nucleotides to siRNA buffer (Dharmacon) to obtain a 50 μM solution. The reaction mixture was heated to 90° C. for 1 min and stored at −20° C. Transfection of RNA oligonucleotides was performed using the RNAi Starter Kit (Qiagen) according to the manufacturer's recommendations, with a final oligonucleotide concentration of 100 nM (for co-transfection of CDK4- and CDK6RNAi, 50 nM of each RNAi was used). After 24 h, the viability was examined and protein expression was assessed by Western blotting using primary antibodies recognizing either CDK4 (monoclonal; 1:250; BD Biosciences), CDK6 (monoclonal; 1:400; Biosource). All experiments were performed in triplicate.

Single Cell Gel Electrophoresis (Comet assay). DNA damage was assessed using the alkaline or neutral single-cell gel electrophoresis (comet assay) method (Kruman, I. I., et al. 2004. Neuron. 41:549-561; Kruman, I. I., et al. 2002. J Neurosci. 22:1752-176229). We used the neutral comet assay which is specific for detecting DSBs (Wojewodzka, M., et al. 2002. Mutat Res. 518:9-20). Following treatment, neurons were scraped and cell suspensions (˜10,000 cells) were embedded into 0.5% low melting agarose on slides (Trevigen, Gaithersburg, Md.). After treatment with cold lysis buffer (Trevigen) containing 1% Triton X-100 and 10% DMSO, the slides were incubated for 1 h in freshly prepared electrophoresis buffer, 300 mM sodium acetate, 100 mM Tris HCl, pH 8.3 (neutral comet assay) or 300 mM NaOH, 1 mM EDTA, pH>13 (alkaline comet assay). Then electrophoresis was performed at 14V and 60 mA for 1 h (neutral comet assay) or at 25V and 300 mA for 30 min (alkaline comet assay) at 8° C., stained with SYBR green (Trevigen), and analyzed using an Olympus BX51 fluorescent microscope and the comet assay image analysis software (Loats Associates Inc.). Nuclei with damaged DNA have the appearance of a comet with a bright head and a tail, where the tail represents the damaged DNA, which is often fragmented and its electrophoretic mobility is consequently greater. Nuclei with undamaged DNA appear round, with no tail. Images of 50 randomly selected cells were analysed from each slide. Data analysis was based on the mean population response or on the distribution of damage among cells. As a control of DSB formation we employed γ-irradiated cortical neurons. Cells were treated with 1 Gy of γ irradiation using an RS 2000 Biological irradiator (Rad Source Technologies, Inc.).

Immunofluorescence. Neurons grown on glass coverslips were fixed in freshly prepared 4% formaldehyde for 30 min at 4° C. and then permeabilized for 10 min in 0.5% Triton X-100 in 1% BSA prepared in PBS, blocked in 1% BSA for 1 h at room temperature, and incubated with the primary antibodies for 1 h at room temperature. We used the following specific antibodies: anti-y-H2AX (monoclonal; 1:500; Upstate Cell Signaling Solutions). As a counterstain, we used PI. Coverslips were mounted with Vectashield mounting medium (Vector Laboratories) and examined with a Nikon Eclipse E800 fluorescence microscope equipped with a Spot digital camera and software.

Statistical analyses. Statistical analyses were performed with Microsoft Excel and p values were obtained using ANOVA and Fisher's post-hoc test. A p value <0.05 was considered significant.

Results

To determine whether G1 cell cycle components are activated in terminally differentiated neurons subjected to DNA damage, the effects of oxidative stress produced by hydrogen peroxide (H₂O₂) were first determined.

H₂O₂ is generated as a product of normal metabolism, is a cell membrane-permeable precursor of various free radicals which have been suggested to contribute to neurodegeneration (Behl, C. 1999. Prog Neurobiol. 57: 301-323), and is known to generate double-strand breaks (DSBs) (Slupphaug, G., et al. 2003. Mutat Res. 531:231-251).

For culturing rat cortical neurons, we employed a previously reported method yielding >99% pure neuronal populations as assessed by immunofluorescent detection of neuron-specific MAP-2 (Kobayashi, S., et al. 2002. Nucleic Acids Res Suppl (2):283-284). By day 4 in culture, MAP-2-positive and GFAP-negative cultures were minimally (3%) contaminated by neuroblasts. The toxic (100 μM) and subtoxic (5 μM) concentrations of H₂O₂ were chosen based on dose-response experiments to assess H₂O₂ toxicity by counting cells with apoptotic nuclei (data not shown), as described earlier (Kruman, I. I., et al. 2002. J. Neurosci. 22:1752-1762).

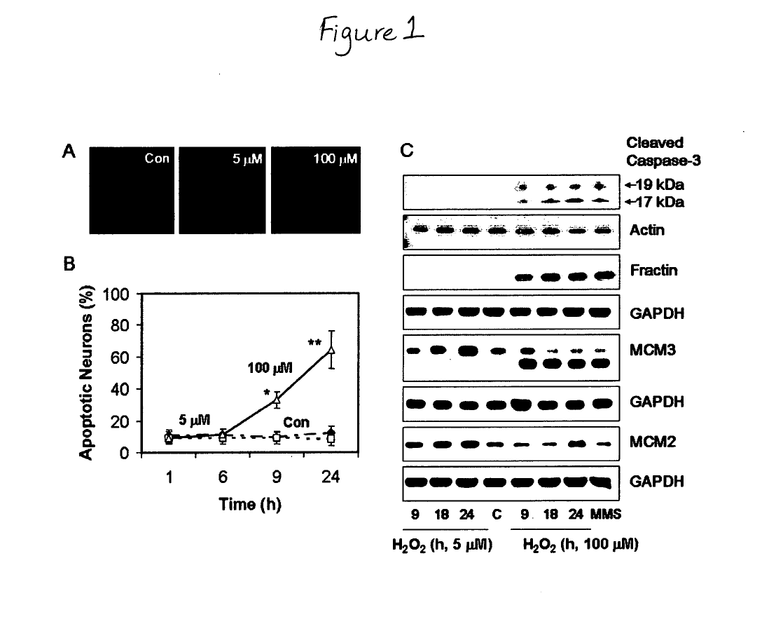

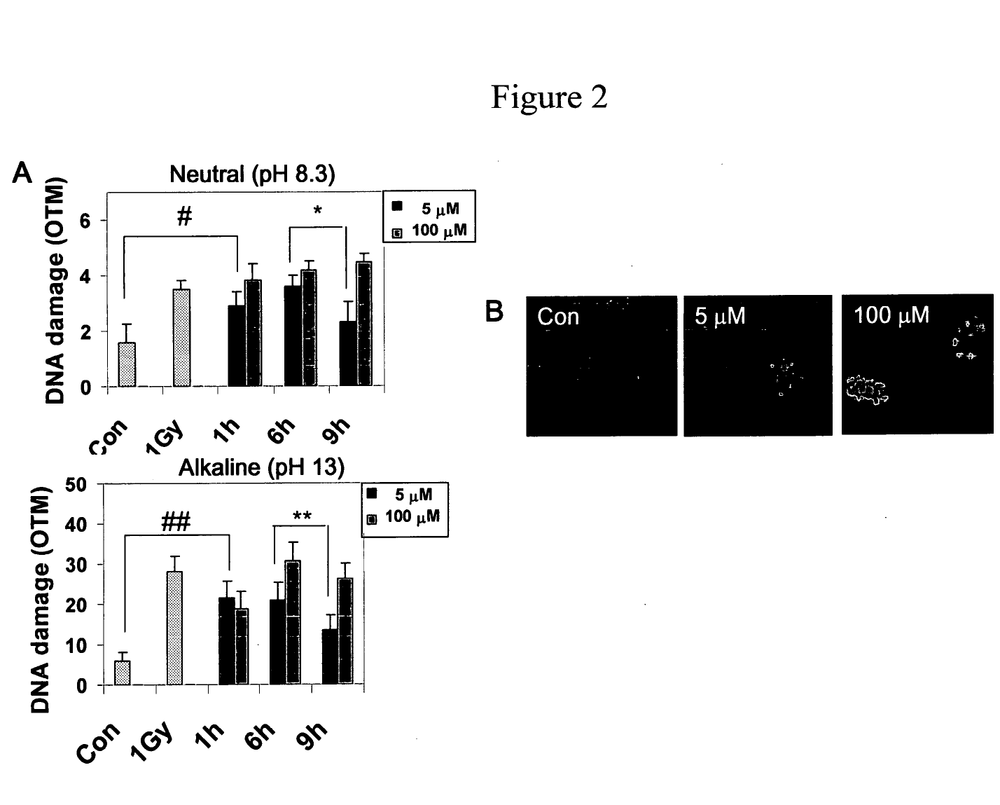

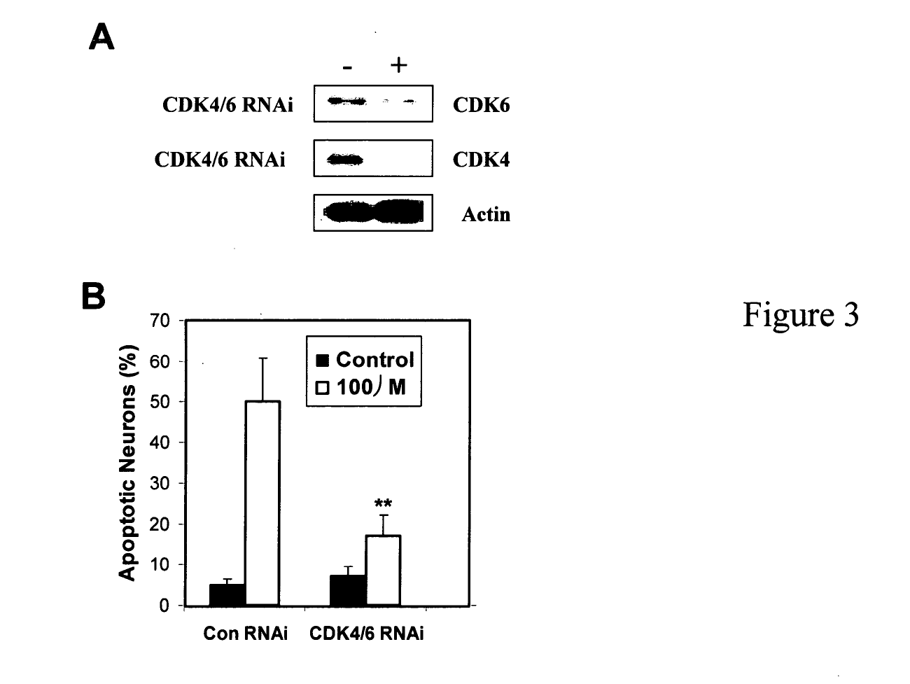

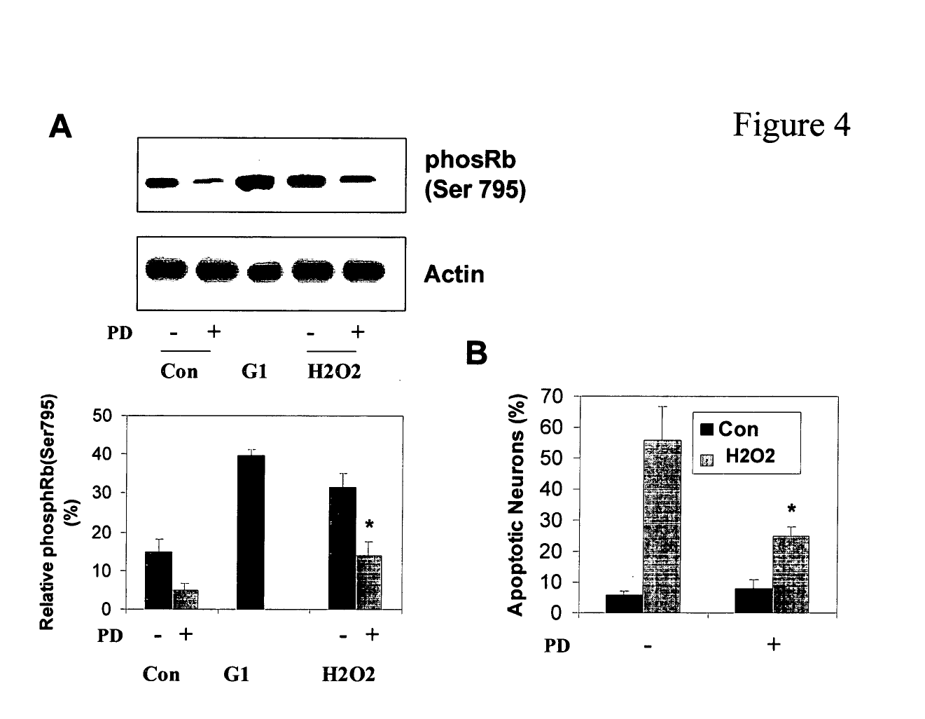

Treatment of cortical neurons with 100 μM H₂O₂ resulted in significant apoptotic death beginning by 9 h following exposure, as evidenced by the appearance of apoptotic nuclei in cultures stained with propidium iodide (FIG. 1A,B) and cleaved caspase-3 intermediates (19 and 17 kDa, which serve as markers of caspase-3 activation during early apoptosis) as assessed by Western blot analysis (FIG. 1C). Extracts from methyl methanesulfonate (MMS)-treated NIH/3T3 cells were used as a positive control (Lakin, N. D. and S. P. Jackson. 1999. Oncogene. 18:7644-7655). Also, the appearance of cleaved β-actin fragment (a 32-kDa C11 terminus fragment) as assessed by Western blot analysis (FIG. 1C) is evidence of caspase3 activation which cleaves cytoskeletal proteins like β-actin during apoptosis (Salvesen, G. S. and V. M. Dixit. 1999. Proc Natl Acad Sci USA. 96: 10964-10967).

Treatment of cortical neurons with 100 μM but not 5 μM H₂O₂ resulted in the cleavage of a nuclear substrate Mcm3 (a 98-kDa fragment), which is typical of early apoptosis, but not Mcm2 (FIG. 1C). These data are consistent with the previous notion that Mcm3, but not other members of the Mcm family of replicative proteins, is an early target in apoptotic proteolysis (Schwab, B. L., et al. 1998. Exp Cell Res. 238:415-421), suggesting that active destruction of Mcm3 inactivates the Mcm complex and serves to prevent untimely DNA replication events during the execution of the cell death program. In contrast to 100 μM H₂O₂, 5 μM H₂O₂ did not induce apoptosis of cortical neurons by 24 h (FIG. 1) or 48 h following exposure (data not shown). Collectively, our results based on using several independent apoptotic markers indicate that 100 μM H₂O₂ is toxic for cultured cortical neurons. In order to test the hypothesis that cell cycle activation accompanies the formation of fatal DSBs in postmitotic neurons, we compared the effects of toxic and subtoxic concentrations of H₂O₂ on DSB formation in cultured cortical neurons.

Changes in DNA damage depend on the concentration of DNA-damaging agent and on the exposure time and reflect a balance between DNA damage and DNA repair. We analyzed the lesions generated by H₂O₂ using the single-cell gel electrophoresis (the comet assay), a sensitive method which has become standard for measuring DNA strand breaks in eukaryotic cells. The assay entails the gel electrophoresis of a small number of cells entrapped in a layer of low-density agarose.

The principle of the assay is based upon the ability of the denaturated DNA fragments to migrate out of the cell during electrophoresis. Nuclei with damaged DNA have the appearance of a comet with a bright head and tail, whereas nuclei with undamaged DNA appear round with no tail.

The ‘alkaline’ (pH 13) version of the comet assay detects a variety of different DNA lesions, including DSB and single strand breaks (SSB), as well as alkaline-labile sites (ALS) and incisions (Collins, A. R. 2004. Mol Biotechnol. 26:249-261). The ‘neutral’ (pH 8.3) version of the comet assay omits the DNA denaturation step, and therefore detects exclusively DSBs as they migrate in the electric field. (Wojewodzka, M., et al. 2002. Mutat Res. 518:9-20). The neutral comet assay has been shown to be a suitable tool for studying the induction and repair of radiation-induced DSBs (Olive, P. L., et al. 1991. Cancer Res. 51:4671-4676; Singh, N. P. and R. E. Stephens. 1997. Mutat Res. 383:167-175; Wojewodzka, M., et al. 2002. Mutat Res. 518:9-20). The neutral comet assay allows the measurement of DNA DSB but, because these lesions are much more toxic and less prevalent (they occur 25 to 40 times less frequently than SSBs), we expected to see much lower levels of DSBs compared to SSBs (Olive, P. L. 1999. Int. J. Radiat. Res. 75:395-405).

To differentiate between double-strand breaks and other types of DNA lesions, we performed two types of the comet assay, alkaline and neutral, and used γ-irradiation as a control of DSB formation, as described earlier (Kruman, I. I., et al. 2004. Neuron. 41:549-561; Morris, E. J., et al. 1999. Biotechniques. 26:282-283, 286-289; Olive, P. L., et al. 1991. Cancer Res. 51:4671-4676; Singh, N. P. and R. E. Stephens. 1997. Mutat Res. 383:167-175; Wojewodzka, M., et al. 2002. Mutat Res. 518:9-20).

Results of the neutral comet assay demonstrate significant increase of DNA damage (notably larger comet tails) in cells exposed to subtoxic 5 μM H₂O₂ (6 h), as illustrated in FIG. 2A. The comparison of DNA damage by the alkaline and neutral comet assays in cortical neurons treated with 5 μM and 100 μM H₂O₂ is shown in FIG. 2A. DNA damage was expressed in Olive Tail Moment (OTM) values, a commonly used parameter which represents the product of the amount of DNA in the tail and the distance between the centers of mass at the head and tail regions.

As expected, in contrast to alkaline version of the assay, the tail moments of the treated cells obtained with the neutral assay were smaller; however, this elevation was significant in cells that were tested 1 and 6 h after treatment with 5 μM H₂O₂ compared to untreated control cells. Importantly, DNA damage significantly decreased in populations exposed to 5 μM but not 100 μM H₂O₂, as assessed by both the alkaline and neutral versions of the comet assay. These findings suggest that DNA damage induced by 5 μM H₂O₂ is repairable, in contrast to DNA damage induced by 100 μM H₂O₂. As an additional measure of DSBs, we monitored the levels of phosphorylation of histone H2AX (γ-H2AX) at serine 139, which occurs at sites surrounding DSBs and can be determined by immunostaining.

Recent reports indicate that the dephosphorylation of γ-H2AX and dispersal of γ-H2AX foci in γ-irradiated cells correlate with DSB repair (MacPhail, S., et al. 2003. Int J Radiat Biol, 79:351-358; Nazarov, I., et al. 2003. Radiat Res. 160:309-317; Rothkamm, K. and M. Lobrich. 2003. Proc Natl Acad Sci USA. 100:5057-5062) and that these parameters provide a quantitative measure of DSB sites (Sedelnikova, O. A., et al. 2002. Radiat Res. 158:486-492). Thus, γ-H2AX foci reveal DSBs (Rothkamm, K. and M. Lobrich. 2003. Proc Natl Acad Sci USA. 100:5057-5062) and can be used as an indicator of the presence of DSBs. In order to relate the effects of treatment with 5 μM and 100 μM H₂O₂ to neuronal death and survival upon DSB DNA damage, we determined the phosphorylation of H2AX in untreated cultures and in cultures exposed to both toxic and subtoxic H₂O₂ concentrations. The extent of H2AX phosphorylation, assessed by immunofluorescence, revealed that the average number of γ-H2AX foci/cell was notably higher in treated cells (FIG. 2B). These data are consistent with the comet assay results (FIG. 2A), as well as with the ensuing apoptosis seen with 100 μM but not 5 μM H₂O₂ (FIG. 1).

Phosphorylation of H2AX, as well as increased OTM in the comet assay can be caused by apoptotic DNA fragmentation (Rogakou, E. P., et al. 2000. J Biol Chem. 275:9390-9395); however, since both DSBs precede apoptotic death, seen in neuronal cultures by 24 h of exposure to 100 μM H₂O₂ (FIG. 1), it appeared that either γ-H2AX formation or DNA damage as determined by the comet assay in those cultures preceded apoptosis. Therefore, 100 μM H₂O₂ produced accumulative breaks that contributed to apoptosis.

As we showed previously, postmitotic neurons undergo DNA damage-initiated apoptosis after cell cycle activation, and cell cycle reentry was essential for the execution of DSB-mediated apoptosis initiated by the classical DSB inducers, y-irradiation and etoposide (Kruman, I. I., et al. 2004. Neuron. 41:549-561).

To confirm the role of cell cycle reentry in DNA damage-initiated neuronal apoptosis, we employed RNA interference (RNAi)-based methods to silence the expression of cyclin dependent kinases CDK4 and CDK6, two CDKs essential for cell cycle activation, and examined the influence of these interventions on H₂O₂-induced apoptosis (Davidson, M. K., et al. 2004. J Biol Chem. 279:50857-50863). Primary cortical neurons were co-transfected with CDK4 and CDK6-targeting siRNA, each representing a cocktail of four siRNA, directed against different regions of the corresponding transcripts (SMARTpool reagents, Dharmacon), or control siRNA. The simultaneous presence of multiple siRNAs elicits more effective gene silencing, while it minimizes off-target effects from each individual siRNA since they are used at lower concentrations (˜12.5 nM). At 24 h after transfection, cells were harvested and the expression of the CDKs was analyzed by Western blot analysis.

FIG. 3A demonstrates the marked reduction in CDK4 and CDK6 levels in cortical neurons at 24 h after transfection. Twenty-four h later, cells were treated with 100 μM H₂O₂ and 18 h (when a significant number of apoptotic cells was expected, FIG. 1B), apoptotic nuclei were assessed. We found a significant reduction in the extent of apoptosis in cells with silenced CDK4 and CDK6 (FIG. 3B). These findings support the notion that cell cycle reentry is essential for the activation of apoptotic program in differentiated neurons exposed to DSB DNA damage.

Treatment with PD 0332991, a highly specific inhibitor of CDK4 and CDK6 (Fry, D. W., et al. 2004. Mol Cancer Ther. 3:1427-1438), resulted in significant reduction in the extent of apoptosis in cells pretreated with PD 0332991 (FIG. 4 B)

Collectively, these observations strongly suggest that activation of the cell cycle machinery is essential in apoptotic signaling in postmitotic neurons.

### Experimental Conclusions

In this Example, evidence is provided that activation of the cell cycle machinery contributes to DNA damage-initiated neuronal apoptosis. To our knowledge, this study is the first to demonstrate that G1 cell cycle components are involved in DNA damage-initiated neuronal apoptosis.

In mitotic cells, the cell cycle machinery is a major contributor to the DNA damage response, a complex defense mechanism whose function is to eliminate the damaged DNA (DNA repair) or, alternatively, to eliminate the damaged cells via apoptosis (Bernstein. C., et al. 2002. Mutat Res. 511:145-178). The latter mechanism ensures that irreparable DNA modifications are not passed on to the progeny of damaged cells. Both DNA repair and apoptosis are coordinated with progression through the cell division cycle, together acting to preserve genomic integrity (Rhind, N. and P. Russell. 2000. Curr Biol. 10:R908-R911). Thus, in proliferating cells, an important role of the DNA damage response is to activate the cell cycle checkpoints (Shiloh, Y. 2003. Nat Rev Cancer. 3:155-168).

In neurons, by contrast, the DNA damage response was not expected to activate the cell cycle checkpoints, due to their postmitotic nature. However, accumulating evidence suggests that neurodegeneration is linked to a paradoxical reentry into the cell cycle (Liu, D. X., and L. A. Greene. 2001. Cell Tissue Res. 305:217-228). There is both in vitro and in vivo evidence of links between DNA damage and cell cycle reentry in dying postmitotic neurons (Klein, J. A., et al. 2002. Nature. 419:367-374, Kruman, I. I., et al. 2004. Neuron. 41:549-561), suggesting that both cell cycle activation and apoptosis are essential components of the DNA damage response. DNA repair is critical for the nervous system, as supported by the fact that hereditary diseases associated with defects in DNA repair defects are associated with neurological abnormalities and progressive neurodegeneration (Rolig, R. L., and P. J. McKinnon. 2000. Trends Neurosci. 23:417-424).

We found that DSBs in postmitotic neurons can arise from oxidative stress produced by H₂O₂ and may result in apoptosis.

Our results indicate that the failure of DSB repair is linked to the onset of apoptosis.

In the above Example, the presence of non-repairable DNA DSBs in surviving neurons was accompanied by an activation of the cell cycle machinery and G0→G1 transition.

Our previous studies demonstrated that the cell cycle was activated in postmitotic neurons committed to DNA damage-initiated apoptosis (Kruman, I. I., et al. 2004. Neuron. 41:549-561). These observations, along with the present work, lead to a scientifically reasonable conclusion that cell cycle machinery plays a central role in the apoptotic signaling of neurons exposed to DNA damage, and that treatment with a highly specific inhibitor of CDK4 and CDK6 (in this instance, PD 0332991) results in significant reduction of the extent of apoptosis in cells pretreated with such an inhibitor.

We claim:

1. A method of ameliorating, treating or preventing neuronal degeneration and/or neuronal death in acute or chronic conditions, comprising administering to a subject in need thereof a therapeutically active amount of an agent that will inhibit one or both of CDK4 and CDK6.

2. The method of claim 1, wherein the agent is an inhibitor of both CDK4 and CDK6.

3. The method of claim 1, wherein the agent has the formula: 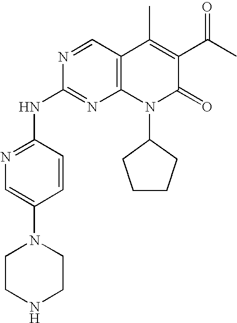
and pharmaceutically acceptable salts or derivatives thereof.

4. The method of claim 1, wherein the acute or chronic condition is: Alzheimer's disease; Parkinson's disease; ischemic states that are due to or result from such conditions as coronary artery bypass graft surgery; global cerebral ischemia due to cardiac arrest; focal cerebral infarction; cerebral hemorrhage; hemorrhage infarction; hypertensive hemorrhage; hemorrhage due to rupture of intracranial vascular abnormalities; subarachnoid hemorrhage due to rupture of intracranial arterial aneurysms; hypertensive encephalopathy; carotid stenosis or blood vessel occlusion leading to cerebral ischemia; cardiogenic thromboembolism; spinal stroke and spinal cord injury; diseases of cerebral blood vessels (such as atherosclerosis and vasculitis); macular degeneration; myocardial infarction; cardiac ischemia; or superaventicular tachyarrhythmia.

5. The method of claim 4, wherein the condition is cerebral stroke due to carotid stenosis or blood vessel occlusion.

6. The method of claim 4, wherein the condition is Alzheimer's disease.
